(19) United States
(12) Patent Application Publication
(10) Pub. No.: US 2026/0316084 A1
(43) Pub. Date: Oct. 15, 2026

(54) KIT FOR MULTIPLEX POLYMERASE CHAIN REACTION

(71) Applicant: Hai Kang Life Corporation Limited, Hong Kong (CN)

(72) Inventor: Cheung Hoi YU, Hong Kong (CN)

(21) Appl. No.: 19/647,257

(22) Filed: Apr. 14, 2026

(30) Foreign Application Priority Data
Apr. 14, 2025 (HK) 320251060309

(51) Int. Cl. B01L 7/00; B01L 3/00

(52) U.S. Cl. CPC B01L 7/52; B01L 3/502715; B01L 3/502738; B01L 2200/0689; B01L 2300/0636; B01L 2300/0645; B01L 2300/087; B01L 2300/1805; B01L 2400/0644

(57) ABSTRACT

The present disclosure discloses a kit for multiplex polymerase chain reaction, including: a hollow module, including a hollow region located at a center and a first chamber and a second chamber located at a periphery of the hollow region; a third chamber for providing a site for the reaction; a rotary valve for driving a sample and/or reagent to be transferred within the chambers; a base, the third chamber being disposed within the base, and a sealing film being disposed on a bottom surface of the base corresponding to the third chamber; the base being used for contacting a heating assembly to accelerate the reaction; a circular hole corresponding to a position below the hollow region being provided in a center of the base and the sealing film, an annular protrusion extending toward the interior of the circular hole being provided on an inner wall of the circular hole, and an annular groove is provided at a bottom of the base on the outer side corresponding to the third chamber, with the heating assembly extending into the annular protrusion and the annular groove; and a detection chip for detecting substances after the multiplex polymerase chain reaction.

The present disclosure claims priority to Chinese Hong Kong Patent Application No. HK32025106030.9, filed on Apr. 14, 2025, and entitled “An Integrated DNA Automatic Amplification and Detection Cartridge Device Incorporating Pre-loaded Reagents and a Nucleic Acid Hybridization Chip,” the entire content of which is incorporated herein by reference.

### TECHNICAL FIELD

The present disclosure relates to the technical field of biological detection devices, and more particularly relates to a kit for automated reaction and detection of multiplex polymerase chain reaction.

### BACKGROUND

With the development of precision medicine and public health surveillance, there is a growing demand for rapid and multiplex detection of pathogens. Conventional real-time quantitative PCR (qPCR) is limited by fluorescence spectral overlap, and the number of channels is typically fewer than six. Although digital PCR offers high sensitivity, the equipment is expensive and difficult to adapt for point-of-care testing (POCT).

Patent CN114739996A provides a disposable virus detection device, in which a piston element drives a main housing sealing film to achieve automatic injection of the stock solution, an amplification chamber sealing element is combined with a flow channel element to accomplish sample amplification, a control device precisely regulates the fluid path, and a detection device integrates an optical sensor to improve reading accuracy, with an overall modular design that facilitates disposable use.

The DNA hybridization chip used in the virus detection device of that patent operates as follows: to detect a specific DNA sequence in a biological sample, capture probes composed of complementary DNA are attached to the surface of a solid-phase carrier; if the biological sample contains the target DNA, the target DNA hybridizes and binds to the complementary DNA fragments. However, this binding process is passive and random, meaning that hybridization efficiency is low and relatively long reaction times are required.

In addition, in above patent, the bottom surface of the base is planar, so that the heating block can only heat the bottom plane, resulting in poor heating performance and low reaction efficiency.

Accordingly, the conventional detection kits still suffer from relatively low heating efficiency, reaction efficiency, and detection efficiency, and there is room for further improvement.

### SUMMARY

In view of the foregoing, in order to overcome the drawbacks of the prior art, an object of the present disclosure is to provide an improved kit suitable for multiplex polymerase chain reaction.

To achieve the above object, the present disclosure adopts the following technical solutions:
a kit for multiplex polymerase chain reaction, used for automated reaction and detection of multiplex PCR, including:
a hollow module including a hollow region located at a center and a first chamber and a second chamber located at a periphery of the hollow region, the first chamber being used for storing a reagent and the second chamber being used for storing a sample;
a third chamber for providing a site for the multiplex polymerase chain reaction; a rotary valve disposed corresponding to the hollow region and used for driving the sample and/or reagent to be transferred within the chambers;
a base, the third chamber being disposed within the base, a sealing film being disposed on a bottom surface of the base corresponding to the third chamber, and the sealing film being used for sealing a bottom of the third chamber; the base being used for contacting a heating assembly to accelerate the reaction; a circular hole corresponding to a position below the hollow region being provided in a center of the base and a center of the sealing film, an annular protrusion extending toward an interior of the circular holes is provided on an inner wall of the circular holes, and an annular groove is provided at a bottom of the base on an outer side corresponding to the third chamber, with the heating assembly extending into the annular protrusion and the annular groove;
a detection chip for detecting substances after the multiplex polymerase chain reaction, a bottom of the detection chip comprising a thermally conductive layer for contacting the heating assembly. The detection chip has a capture probe for capturing target DNA fragments and accelerates the hybridization process of DNA through electric-field assistance. Preferably, the detection chip is an electric field assisted microarray hybridization chip.

The heating assembly comprises a heating block, wherein the heating block includes a first extension portion that extends into the circular hole and extends to the annular protrusion, and a step portion located below the annular protrusion; the first extension portion and the step portion are in contact with the inner wall of the circular hole, and a top of the first extension portion is higher than the annular protrusion; the heating block includes a second extension portion that extends into the annular groove, and the second extension portion is in contact with the inner wall of the annular groove, so as to realize three-dimensional contact between the heating assembly and the base and increase the contact area.

According to some preferred embodiments of the present disclosure, the rotary valve comprises a valve disc and a valve barrel extending upward from the valve disc, wherein the valve disc is located between the hollow module and the third chamber, the valve barrel is located within the hollow region, a receiving cavity is arranged within the valve barrel, a plunger is disposed within the receiving cavity, a valve inlet is provided in an upper portion of the valve disc, and a valve outlet is provided in a lower portion of the valve disc; the valve inlet is used for communicating the second chamber or the first chamber with the receiving cavity, and the valve outlet is used for communicating the third chamber with the receiving cavity. A communication hole is provided in the bottom of the first chamber, and the valve disc of the rotary valve rotates so as to place the valve inlet in communication with the communication hole, thereby drawing a substance in the first chamber into the receiving cavity or injecting a substance in the receiving cavity into the first chamber.

Preferably, a communication tube is disposed in the first chamber corresponding to the communication hole, one end of the communication tube is disposed within the communication hole, and the other end of the communication tube extends into the first chamber.

Preferably, the communication tube comprises a first segment, a second segment, and a third segment arranged in sequence, wherein one end of the first segment is inserted into the communication hole and the other end of the first segment extends upward, the second segment extends horizontally from an upper end of the first segment, and the third segment extends downward from the second segment and is close to the bottom of the first chamber. The first segment and the third segment are vertically arranged; the first segment is inserted into the communication hole to prevent the reagent from leaking downward through the communication hole, and the third segment is close to the bottom of the first chamber so as to draw the reagent within the first chamber. A distance between a lower end of the third segment and the bottom of the first chamber is 0-1 mm.

Preferably, all of the communication holes are located on a same circumference, and a projection of the third segment on a horizontal plane is located on the circumference. The communication holes (the circumference) are correspondingly disposed at the lowest position within the first chamber, and the third segment is also correspondingly disposed at this lowest position, so as to avoid or reduce residue of the reagent within the first chamber.

Further, a lower end opening of the third segment is obliquely arranged, so as to allow the substance within the first chamber to enter the receiving cavity through the communication tube.

Further, the kit is provided with a fourth chamber for detection and a fifth chamber for receiving post-detection waste liquid, a discharge pipe is provided between the fourth chamber and the fifth chamber, and an isolation hole is provided on a side of the kit corresponding to the discharge pipe, whereby the discharge pipe is squeezed through the isolation hole to close a passage between the fourth chamber and the fifth chamber.

Owing to the adoption of the above technical solutions, compared with the conventional art, the beneficial effects of the present disclosure are as follows: in the kit for multiplex polymerase chain reaction of the present disclosure, the sealing film is configured in an annular shape so that the heating block extends into the circular hole, and at the same time, by providing the annular protrusion and the annular groove and configuring the heating block into a corresponding shape, the contact area between the base and the heating block is increased, thereby improving the heating effect and reaction speed and rendering PCR amplification more efficient; meanwhile, a thermally conductive layer is provided at the bottom of the detection chip to control the detection temperature, and an electric field assisted microarray hybridization chip is employed, whereby a DNA hybridization process can be accelerated by applying a specific electric field, so that the capture of DNA fragments is changed from passive random capture to active capture, thereby accelerating the detection process and enhancing the detection capability for low-concentration samples.

### BRIEF DESCRIPTION OF THE DRAWINGS

FIG. 1 and FIG. 2 are schematic perspective structural diagrams of a kit in an embodiment of the present disclosure from different viewing angles;

FIG. 3 is a schematic perspective structural diagram of the kit in an embodiment of the present disclosure with the cover hidden;

FIG. 4 is a schematic top structural diagram of the kit in an embodiment of the present disclosure with the cover hidden;

FIG. 5 is a schematic cross-sectional structural diagram along A-A in FIG. 4;

FIG. 6 is a schematic front structural diagram of the communication tube in an embodiment of the present disclosure;

FIG. 7 and FIG. 8 are schematic perspective structural diagrams of the base in an embodiment of the present disclosure from different viewing angles;

FIG. 9 is a schematic top structural diagram of the base in an embodiment of the present disclosure;

FIG. 10 is a schematic cross-sectional structural diagram along B-B in FIG. 9;

FIG. 11 is a schematic cross-sectional structural diagram of the base mated with the heating block in an embodiment of the present disclosure;

FIG. 12 is a schematic perspective structural diagram of the rotary valve in an embodiment of the present disclosure;

FIG. 13 is a schematic top structural diagram of the rotary valve in an embodiment of the present disclosure;

FIG. 14 is a schematic cross-sectional structural diagram along C-C in FIG. 13;

In the drawings: main body—1, first cover—21, second cover—22, hollow module—3, hollow region—31, first chamber—32, second chamber—33, third chamber—34, third chamber inlet—341, third chamber outlet—342, rotary valve—5, valve disc—51, valve barrel—52, receiving cavity—53, plunger—54, valve inlet—55, valve outlet—56, exhaust passage—57, communication hole—61, communication tube—62, first segment—71, second segment—72, third segment—73, base—8, circular hole—81, annular groove—82, annular protrusion—83, detection assembly—9, detection chip—91, fourth chamber—92, fifth chamber—10, sealing film—11, communication flow channel—12, kit—13, isolation hole—131, heating block—14, first extension portion—141, second extension portion—142, step portion—143, discharge pipe—15.

### DETAILED DESCRIPTION

The technical solutions of the present disclosure will be further described below in conjunction with the accompanying drawings and preferred embodiments.

In a conventional kit 13, on the one hand, because the communication hole 61 at the bottom of the first chamber 32 is formed integrally by injection molding, it has a relatively large diameter (about 1 mm), and at the same time, the valve disc 51 of the rotary valve 5 generates a slight negative pressure during rotation; under the gravity of the reagent and the negative pressure generated when the valve disc 51 rotates, the reagent leaks downward, and the leaked reagent is carried by the valve disc 51 to other chambers, causing cross-contamination. On the other hand, the sealing film 11 of the existing kit 13 is integral, and the bottom surface of the base 8 corresponding to the third chamber 34 is planar, so that the heating block 14 can only heat the third chamber 34 through the sealing film 11, resulting in unsatisfactory heating effect and low efficiency.

In order to solve the above problems, in a kit 13 of the present disclosure, on the one hand, a communication tube 62 is provided in the first chamber 32 to prevent the reagent from leaking downward through the communication hole 61 and causing contamination; on the other hand, the structure of the base 8 and the sealing film 11 is improved to enhance the heating effect.

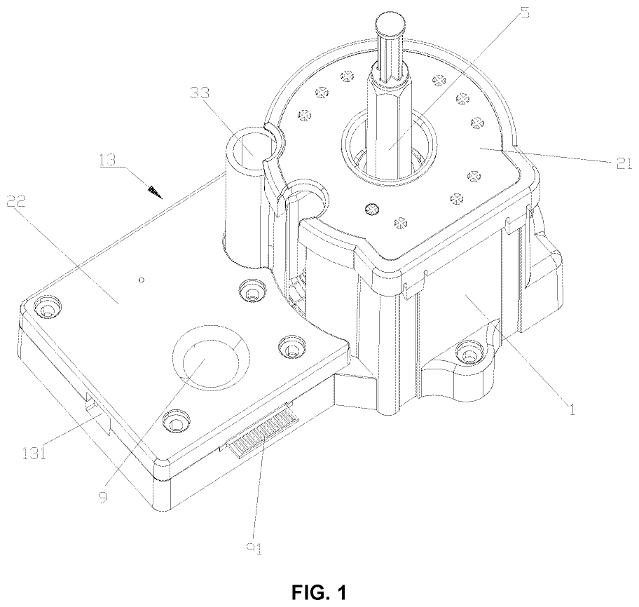

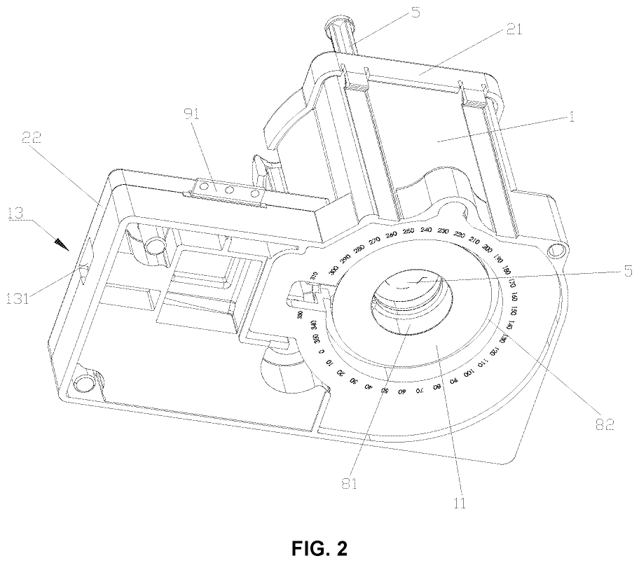

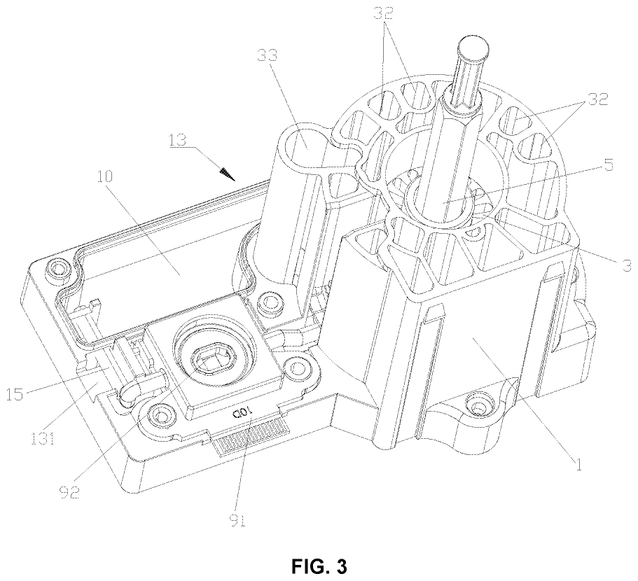

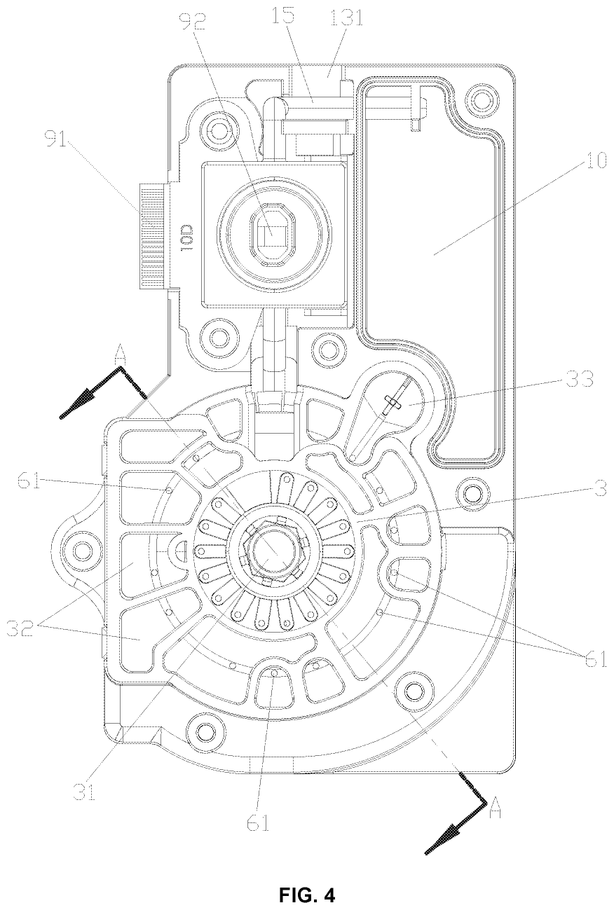

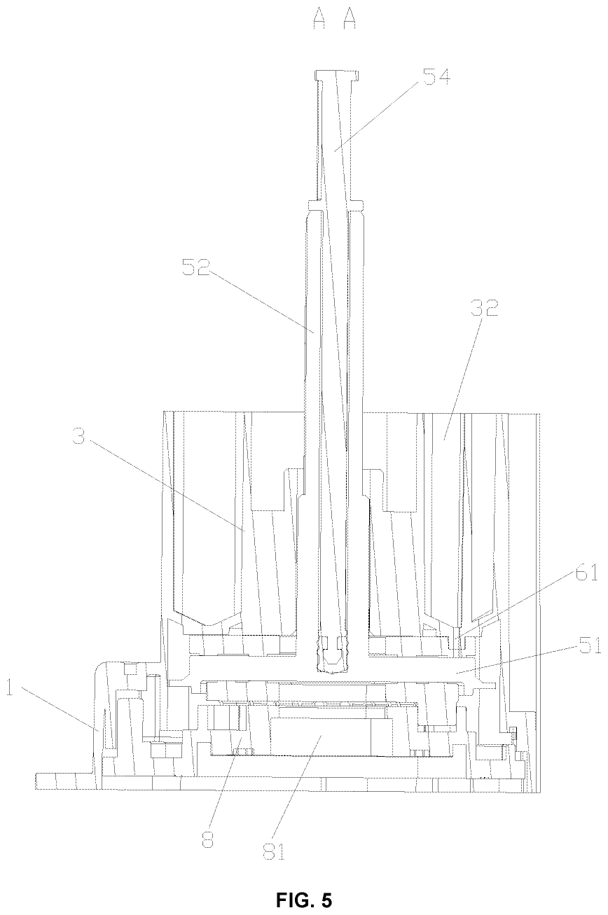

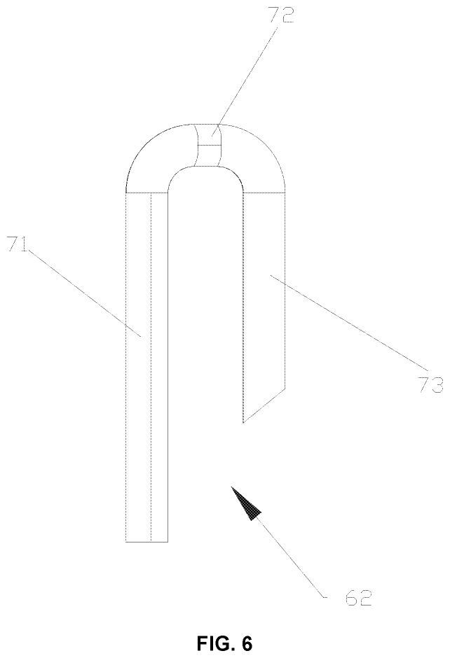

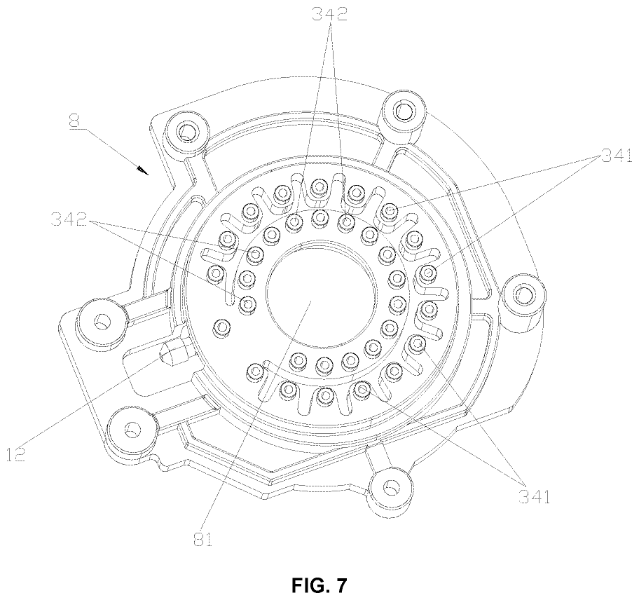

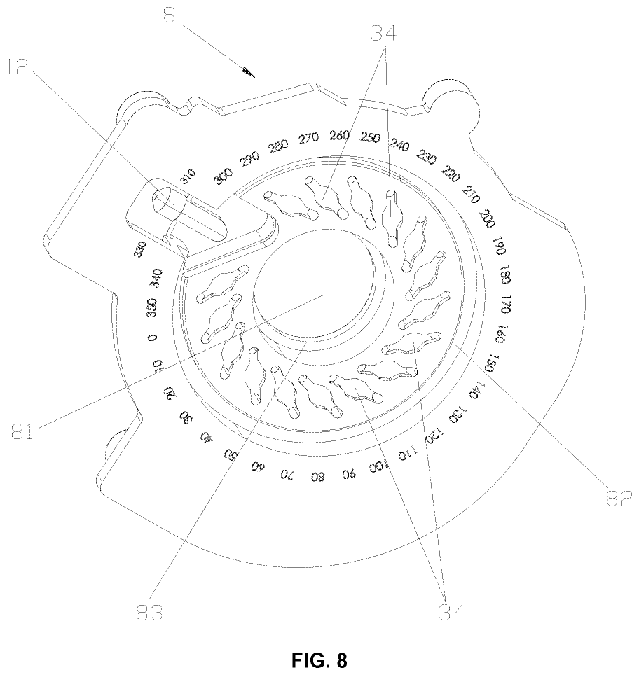

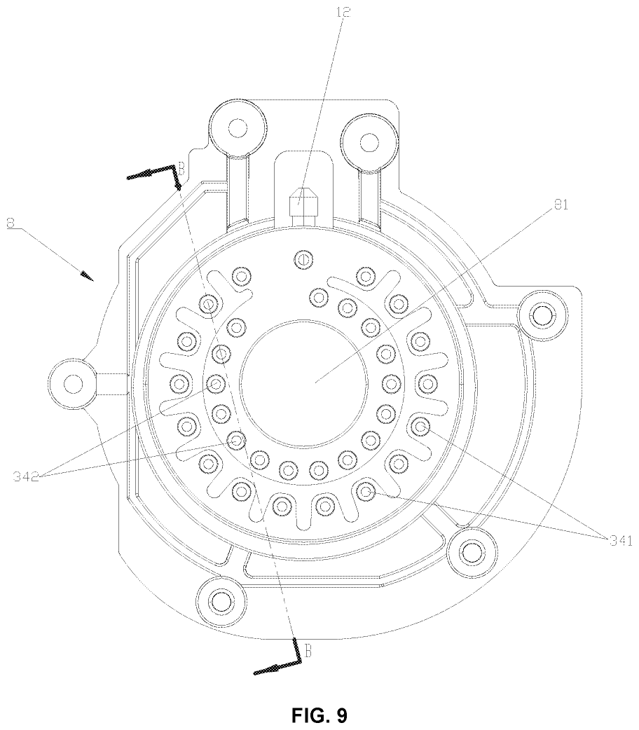

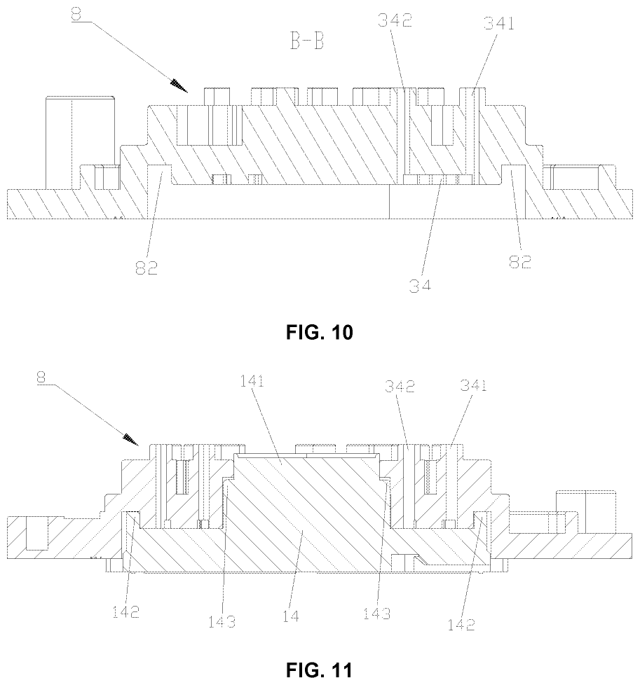

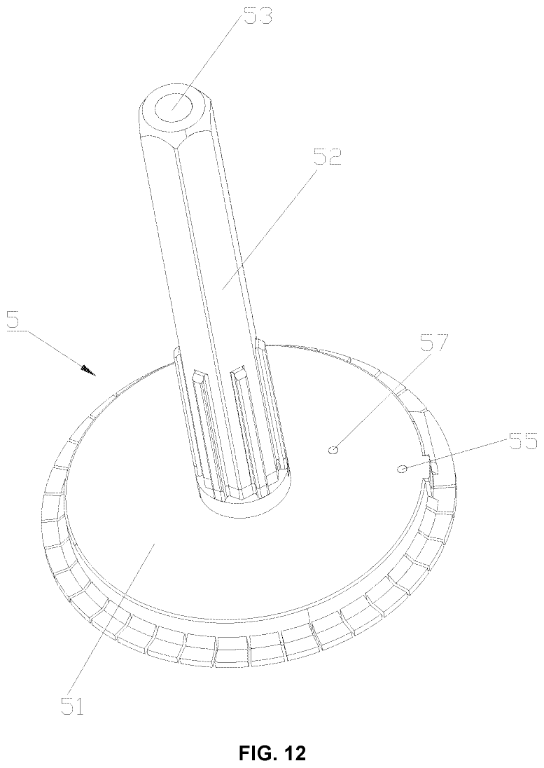

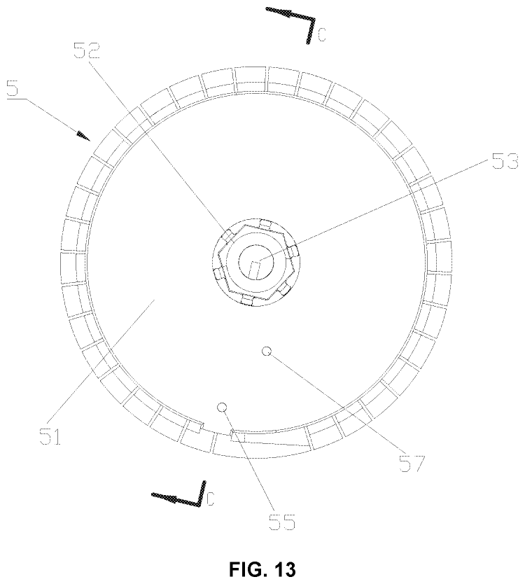

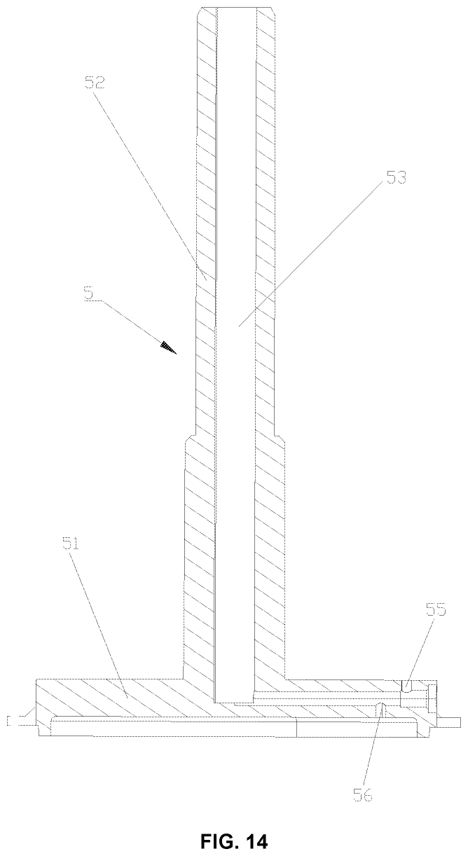

Specifically, as shown in FIG. 1 to FIG. 14, the kit 13 for multiplex polymerase chain reaction of the present embodiment is used for automated reaction and detection of multiplex PCR, and comprises a main body 1, a first cover 21, a second cover 22, a detection assembly 9, a base 8, and a rotary valve 5. The main body 1 comprises a hollow module 3, the first cover 21 is disposed above the hollow module 3, and the base 8 is disposed below the hollow module 3. The detection assembly 9 is disposed on the main body 1, the first cover 21 covers the detection assembly 9, and a fifth chamber 10 for receiving post-detection waste liquid is further disposed on the main body 1 corresponding to the detection assembly 9. The detection assembly 9 comprises a fourth chamber 92 and a detection chip 91, and a discharge pipe 15 made of a flexible material is disposed between the fourth chamber and the fifth chamber. The detection chip 91 has reaction sites in a predetermined configuration for capturing target DNA fragments expected to be complementary by means of capture fragment base sequences (capture probes).

To facilitate detection and to isolate the waste liquid from the substance to be detected, an isolation hole 131 is provided on a side of the kit corresponding to the discharge pipe, and the discharge pipe 15 is squeezed through the isolation hole 131 to close the passage between the fourth chamber 92 and the fifth chamber 10.

The hollow module 3 comprises a hollow region 31 located at the center and a first chamber 32 and a second chamber 33 located at the periphery of the hollow region 31, wherein the second chamber 33 is used for storing a sample and the first chamber 32 is used for storing a reagent. The rotary valve 5 is disposed corresponding to the hollow region 31 and is used for driving the sample and/or reagent to be transferred within the chambers (the first chamber 32, the second chamber 33, the third chamber 34, the fourth chamber 92, and the fifth chamber 10).

A third chamber 34 for providing a reaction site is disposed within the base 8. A sealing film 11 is disposed on the bottom surface of the base 8 corresponding to the third chamber 34, and the sealing film 11 is used for sealing the bottom of the third chamber 34; the base 8 is used for contacting a heating assembly to accelerate the reaction. In the use of the kit 13, the base 8 and the third chamber 34 are heated to accelerate the reaction of the substance within the third chamber 34. A communication flow channel 12 for communicating with the detection assembly 9 is further provided on the base 8, so as to deliver the substance to be detected (the amplification product) to the detection assembly 9 for detection.

A circular hole 81 corresponding to a position below the hollow region 31 is provided in the middle of the base 8 and the sealing film 11, and an annular protrusion 83 extending toward the interior of the circular hole 81 is provided on the inner wall of the circular hole 81. The heating assembly comprises a heating block 14, which extends into the circular hole 81 and contacts the inner wall of the circular hole 81, with the top of the heating block 14 being higher than the annular protrusion 83. Meanwhile, an annular groove 82 is provided at the bottom of the base 8 on the outer side corresponding to the third chamber 34, and the heating block 14 extends into the annular groove 82 and contacts the inner wall of the annular groove 82. In the present embodiment, the sealing film 11 is configured in an annular shape so that the heating block 14 can extend into the circular hole 81; at the same time, by providing the annular protrusion 83 and the annular groove 82 and configuring the heating block 14 into a corresponding shape, the contact area between the base 8 and the heating block 14 is increased, thereby improving the heating effect and reaction speed and making PCR amplification more efficient.

Specifically, the heating block 14 comprises a first extension portion 141, a step portion 143, and a second extension portion 142. The first extension portion 141 extends into the circular hole and extends to the annular protrusion, with the top of the first extension portion 141 being higher than the annular protrusion; the step portion 143 is located below the annular protrusion; the second extension portion 142 extends into the annular groove; the first extension portion 141 and the step portion 143 contact the inner wall of the circular hole, and the second extension portion 142 contacts the inner wall of the annular groove.

The rotary valve 5 comprises a valve disc 51, a valve barrel 52 extending upward from the valve disc 51, and a plunger 54 disposed within the valve barrel 52. The valve disc 51 is located between the hollow module 3 and the base 8, the valve barrel 52 is located within the hollow region 31, a receiving cavity 53 is provided within the valve barrel 52, and the plunger 54 is disposed within the receiving cavity 53. A valve inlet 55 is provided in the upper portion of the valve disc 51, and a valve outlet 56 is provided in the lower portion of the valve disc 51. The valve inlet 55 is used for communicating the second chamber 33 or the first chamber 32 with the receiving cavity 53, and the valve outlet 56 is used for communicating the third chamber inlet 341 with the receiving cavity 53. An exhaust passage 57 penetrating the thickness of the valve disc 51 is further provided in the valve disc 51, and the exhaust passage 57 communicates with the third chamber outlet 342.

A communication hole 61 is provided in the bottom of the first chamber 32, and the communication hole 61 serves as the passage communicating the first chamber 32 with the rotary valve 5. The valve disc 51 of the rotary valve 5 rotates to place the valve inlet 55 in communication with different communication holes 61, so as to draw the substance in the first chamber 32 into the receiving cavity 53 or inject the substance in the receiving cavity 53 into the first chamber 32; at the same time, the valve outlet 56 is brought into communication with different third chamber inlets 341 and the receiving cavity 53, or into communication with the communication flow channel 12. In the present embodiment, a communication tube 62 is provided in the first chamber 32 corresponding to the communication hole 61, with one end of the communication tube 62 disposed within the communication hole 61 and the other end extending into the first chamber 32. In the present embodiment, the communication tube 62 is provided on the communication hole 61, and the reagent in the first chamber 32 is drawn through the communication tube 62, thereby preventing the reagent from leaking downward through the communication hole 61 and causing contamination.

Specifically, the communication tube 62 comprises a first segment 71, a second segment 72, and a third segment 73 arranged in sequence. One end of the first segment 71 is inserted into the communication hole 61, and the other end of the first segment 71 extends upward; the second segment 72 extends horizontally from the upper end of the first segment 71; and the third segment 73 extends downward from the second segment 72 and is close to the bottom of the first chamber 32. The first segment 71 and the third segment 73 are vertically arranged; the first segment 71 is inserted into the communication hole 61 to prevent the reagent from leaking downward through the communication hole 61, and the third segment 73 is close to the bottom of the first chamber 32 so as to draw the reagent within the first chamber 32.

All of the communication holes 61 are located on a same circumference, and preferably, the projection of the third segment 73 on a horizontal plane is also located on this circumference. The communication holes 61 (this circumference) are correspondingly disposed at the lowest position within the first chamber 32, and the third segment 73 is also correspondingly disposed at this lowest position, so as to avoid or reduce residue of the reagent within the first chamber 32. The lower end opening of the third segment 73 is obliquely arranged to allow the substance within the first chamber 32 to enter the receiving cavity 53 through the obliquely arranged opening. The distance between the lower end of the third segment 73 and the bottom of the first chamber 32 is 0 mm, i.e., the two are in contact; at the same time, the substance within the first chamber 32 is drawn by means of the obliquely arranged lower end opening of the third segment 73.

In a conventional hybridization chip, the target DNA and the capture probes (capture DNA fragments complementary to the target DNA) interact passively and randomly, resulting in low hybridization efficiency and long reaction times. In the present embodiment, the detection chip 91 is further preferably an electric field assisted microarray hybridization chip, which can accelerate the DNA hybridization process through electric-field assistance. The electric field assisted microarray hybridization chip is an existing detection chip, such as the device and method for detecting nucleic acid in a biological sample disclosed in patent CN1867681A or the biochip for biomolecule detection or sequencing and the manufacturing method thereof disclosed in patent CN106757376A, and details thereof are omitted here.

The kit 13 for multiplex polymerase chain reaction of the present disclosure adopts an electric field assisted microarray hybridization chip, whereby a DNA hybridization process can be accelerated by applying a specific electric field, so that the capture of DNA fragments is changed from passive random capture to active capture, thereby accelerating the detection process and enhancing the detection capability for low-concentration samples. The detection method mainly comprises: introducing a sample into the second chamber of the kit; switching by rotation of the rotary valve to connect the second chamber, the first chamber, and the third chamber; driving the reagent to mix and be dispensed into the third chamber by means of the rotary valve and the plunger; performing PCR amplification in the third chamber; introducing the amplification product to a DNA hybridization microarray chip and capturing target DNA fragments via electric field assisted hybridization technology; and detecting the hybridization efficiency and quantifying the target gene by means of an optical or electronic detection system. The specific detection process and detection method are as described in patent CN114739996A (a disposable virus detection device) and patent HK30118795A (an integrated DNA automatic amplification and detection kit device incorporating pre-loaded reagents and a nucleic acid hybridization chip), and details thereof are omitted here. Meanwhile, a thermally conductive layer for contacting a heating assembly, such as a silicon-based thermally conductive material, may be provided at the bottom of the detection chip; this heating assembly is provided independently of the heating assembly corresponding to the base so as to control the detection temperature, thereby ensuring the efficiency and accuracy of the detection.

In the present embodiment, the kit 13 integrates a typical molecular detection process (including sample introduction, reagent dispensing, PCR amplification, target gene capture, and subsequent detection) into an automated kit 13. With the aid of an instrument system (such as the assay cartridge diagnostic detection equipment disclosed in patent CN117761050A, of which the heating assembly is a part), the kit 13 can automatically complete all experimental processes. The detection chip 91 of the kit 13 can capture a complementary pathogen target gene (DNA fragment), and using an appropriate detection method (for example, fluorescence labeling, current detection, optical imaging, or impedance measurement), the pathogen target gene can be accurately quantified. During nucleic acid hybridization, the DNA hybridization process can be accelerated by applying an electric field, so that the capture of DNA fragments is changed from passive random capture to active capture, thereby accelerating the detection process and enhancing the detection capability for low-concentration samples.

The present disclosure will now be described in conjunction with various examples described below and various combinations thereof.
1. An example provides a kit for multiplex polymerase chain reaction, comprising:
   a hollow module, comprising a hollow region located at a center and a first chamber and a second chamber located at an outer periphery of the hollow region, wherein the first chamber is used for storing a reagent and the second chamber is used for storing a sample;
   a third chamber for providing a site for the multiplex polymerase chain reaction;
   a rotary valve disposed corresponding to the hollow region and used for driving the sample and/or reagent to be transferred within the chambers;
   a detection chip for detecting substances after the multiplex polymerase chain reaction.
2. The kit provided according to Example 1, wherein the kit comprises a base, the third chamber is disposed within the base, a sealing film is disposed on a bottom surface of the base corresponding to the third chamber, the sealing film is used for sealing a bottom of the third chamber, and the base is used for contacting a heating assembly to accelerate the reaction.
3. The kit provided according to any of the preceding examples, wherein a circular hole corresponding to a position below the hollow region is provided in the center of the base and in the center of the sealing film, an annular protrusion extending toward the interior of the circular hole is provided on the inner wall of the circular hole, the heating assembly comprises a heating block, the heating block comprises a first extension portion that extends into the circular hole and extends to the annular protrusion, and a step portion located below the annular protrusion, the first extension portion and the step portion contact the inner wall of the circular hole, and the top of the first extension portion is higher than the annular protrusion.
4. The kit provided according to any of the preceding examples, wherein an annular groove is provided at the bottom of the base on the outer side corresponding to the third chamber, the heating assembly extends into the annular protrusion and the annular groove, the heating block comprises a second extension portion extending into the annular groove, and the second extension portion contacts the inner wall of the annular groove.
5. The kit provided according to any of the preceding examples, wherein the bottom of the detection chip has a thermally conductive layer for contacting a heating assembly, the detection chip is an electric field assisted microarray hybridization chip, the detection chip has a capture probe for capturing target DNA fragments, and a DNA hybridization process is accelerated by electric field assistance.
6. The kit provided according to any of the preceding examples, wherein the rotary valve comprises a valve disc and a valve barrel extending upward from the valve disc, the valve disc is located between the hollow module and the third chamber, the valve barrel is located within the hollow region, a receiving cavity is provided within the valve barrel, a plunger is disposed within the receiving cavity, a valve inlet is provided in the upper portion of the valve disc, a valve outlet is provided in the lower portion of the valve disc, the valve inlet is used for communicating the second chamber or the first chamber with the receiving cavity, and the valve outlet is used for communicating the third chamber with the receiving cavity.
7. The kit provided according to any of the preceding examples, wherein a communication hole is provided in the bottom of the first chamber, and the valve disc of the rotary valve rotates so as to place the valve inlet in communication with the communication hole, thereby drawing a substance in the first chamber into the receiving cavity, or injecting a substance in the receiving cavity into the first chamber.
8. The kit provided according to any of the preceding examples, wherein a communication tube is provided in the first chamber corresponding to the communication hole, one end of the communication tube is disposed within the communication hole, and the other end of the communication tube extends into the first chamber.
9. The kit provided according to any of the preceding examples, wherein the communication tube comprises a first segment, a second segment, and a third segment arranged in sequence, one end of the first segment is inserted into the communication hole and the other end of the first segment extends upward, the second segment extends horizontally from an upper end of the first segment, and the third segment extends downward from the second segment and is close to the bottom of the first chamber.
10. The kit provided according to any of the preceding examples, wherein a distance between the lower end of the third segment and the bottom of the first chamber is 0-1 mm.
11. The kit provided according to any of the preceding examples, wherein all of the communication holes are located on a same circumference, and a projection of the third segment on a horizontal plane is located on the circumference.
12. The kit provided according to any of the preceding examples, wherein a lower end opening of the third segment is obliquely arranged, so as to allow the substance within the first chamber to enter the receiving cavity through the communication tube.
13. The kit provided according to any of the preceding examples, wherein the kit is provided with a fourth chamber for detection and a fifth chamber for receiving post-detection waste liquid, a discharge pipe is provided between the fourth chamber and the fifth chamber, and an isolation hole is provided on a side of the kit corresponding to the discharge pipe, whereby the discharge pipe is squeezed through the isolation hole to close the passage between the fourth chamber and the fifth chamber.

Finally, it should be noted that the above embodiments are merely for illustrating the technical solutions of the present disclosure and are not intended to limit the same. Although the present disclosure has been described in detail with reference to the above embodiments, those of ordinary skill in the art should understand that modifications or equivalent substitutions may still be made to the specific embodiments of the present disclosure, and any modifications or equivalent substitutions that do not depart from the spirit and scope of the present disclosure shall be encompassed within the protection scope of the claims of the present disclosure.

What is claimed is:

1. A kit for multiplex polymerase chain reaction, comprising:
a hollow module comprising a hollow region located at a center and a first chamber and a second chamber located at an outer periphery of the hollow region, the first chamber being used for storing a reagent and the second chamber being used for storing a sample;
a third chamber for providing a site for the multiplex polymerase chain reaction;
a rotary valve disposed corresponding to the hollow region and used for driving the sample and/or reagent to be transferred within the chambers;
a base, the third chamber being disposed within the base, a sealing film being disposed on a bottom surface of the base corresponding to the third chamber, and the sealing film being used for sealing a bottom of the third chamber; the base being used for contacting a heating assembly to accelerate the reaction; a circular hole corresponding to a position below the hollow region being provided in a center of the base and a center of the sealing film, an annular protrusion extending toward an interior of the circular holes is provided on an inner wall of the circular holes, and an annular groove is provided at a bottom of the base on an outer side corresponding to the third chamber, with the heating assembly extending into the annular protrusion and the annular groove;
a detection chip for detecting substances after the multiplex polymerase chain reaction, a bottom of the detection chip comprising a thermally conductive layer for contacting the heating assembly.

2. The kit according to claim 1, wherein the heating assembly comprises a heating block, the heating block comprises a first extension portion that extends into the circular hole and extends to the annular protrusion, and a step portion located below the annular protrusion, the first extension portion and the step portion contact the inner wall of the circular hole, and a top of the first extension portion is higher than the annular protrusion.

3. The kit according to claim 1, wherein the heating block comprises a second extension portion extending into the annular groove, and the second extension portion contacts the inner wall of the annular groove.

4. The kit according to claim 1, wherein the detection chip is an electric field assisted microarray hybridization chip, the detection chip has a capture probe for capturing target DNA fragments, and a DNA hybridization process is accelerated by electric-field assistance.

5. The kit according to claim 1, wherein the rotary valve comprises a valve disc and a valve barrel extending upward from the valve disc, the valve disc is located between the hollow module and the third chamber, the valve barrel is located within the hollow region, a receiving cavity is arranged within the valve barrel, a plunger is disposed within the receiving cavity, a valve inlet is provided in an upper portion of the valve disc, a valve outlet is provided in a lower portion of the valve disc, the valve inlet is used for communicating the second chamber or the first chamber with the receiving cavity, and the valve outlet is used for communicating the third chamber with the receiving cavity.

6. The kit according to claim 5, wherein a communication hole is provided in a bottom of the first chamber, and the valve disc of the rotary valve rotates so as to place the valve inlet in communication with the communication hole, thereby drawing a substance in the first chamber into the receiving cavity, or injecting a substance in the receiving cavity into the first chamber.

7. The kit according to claim 6, wherein a communication tube is provided in the first chamber corresponding to the communication hole, one end of the communication tube is disposed within the communication hole, and the other end of the communication tube extends into the first chamber.

8. The kit according to claim 7, wherein the communication tube comprises a first segment, a second segment, and a third segment arranged in sequence, one end of the first segment is inserted into the communication hole and the other end of the first segment extends upward, the second segment extends horizontally from an upper end of the first segment, and the third segment extends downward from the second segment and is close to the bottom of the first chamber.

9. The kit according to claim 8, wherein a distance between a lower end of the third segment and the bottom of the first chamber is 0-1 mm.

10. The kit according to claim 8, wherein the kit comprises a plurality of communication holes, all of the communication holes are located on a same circumference, and a projection of the third segment on a horizontal plane is located on the circumference.

11. The kit according to claim 8, wherein a lower end opening of the third segment is obliquely arranged, so as to allow the substance within the first chamber to enter the receiving cavity through the communication tube.

12. The kit according to claim 1, wherein the kit is provided with a fourth chamber for detection and a fifth chamber for receiving post-detection waste liquid, a discharge pipe is provided between the fourth chamber and the fifth chamber, and an isolation hole is provided on a side of the kit corresponding to the discharge pipe, whereby the discharge pipe is squeezed through the isolation hole to close a passage between the fourth chamber and the fifth chamber.
